(19) United States
(12) Patent Application Publication
(10) Pub. No.: US 2006/0009697 A1
(43) Pub. Date: Jan. 12, 2006

(54) WIRELESS, INTERNET-BASED SYSTEM FOR MEASURING VITAL SIGNS FROM A PLURALITY OF PATIENTS IN A HOSPITAL OR MEDICAL CLINIC

(75) Inventors: Matthew John Banet, Del Mar, CA (US); Henk Visser II., San Diego, CA (US)

Correspondence Address: MATTHEW J. BANET, 6540 LUSK BLVD., C200, SAN DIEGO, CA, 92121, US

(73) Assignee: TRIAGE WIRELESS, INC., San Diego, CA, CA (US)

(21) Appl. No.: 11/162,719

(22) Filed: Sep. 20, 2005

Related U.S. Application Data
(63) Continuation-in-part of application No. 10/709,014, filed on Apr. 7, 2004.

(51) Int. Cl. A61B 5/02

(52) U.S. Cl. 600/485; 128/903

(57) ABSTRACT

The invention provides system for measuring vital signs from multiple patients, typically in an in-hospital setting. The system features a body-worn vital sign monitor that includes: i) a sensor configured as a patch that measures electrical and optical signals from a patient; ii) a controller featuring a microprocessor that receives and processes the electrical and optical signals to determine the patient's vital sign information, including blood pressure; and iii) a first short-range wireless component that wirelessly transmits a packet comprising the vital sign information to an external receiver. A portable, wireless computer (e.g., a PDA, cellular telephone, or a laptop computer) communicates with the body-worn module. The wireless computer includes: i) a second short-range wireless component that receives the vital sign information and displays it; and ii) a long-range wireless transmitter that transmits the vital sign information over a wireless network. The system also includes an Internet-based system that receives the vital sign information from the wireless network, and avails this to medical professionals through an in-hospital information system.

### BACKGROUND OF THE INVENTION

The present invention relates to a device, method, and system for measuring vital signs, particularly blood pressure.

### DESCRIPTION OF RELATED ART

Personal digital assistants (‘PDAs’) are currently used in hospitals and medical clinics to, e.g., record notes, collect patient information, and generate prescriptions. Some PDAs, such as Palm's Treo 650 and Audiovox's PPC 6600/6601, include long-range wireless transmitters (e.g., a CDMA modem) that allow them to wirelessly transmit and receive information and ultimately and communicate wirelessly with in-hospital information systems. For example, the above-mentioned PDAs can run software programs that wirelessly connect through the Internet to the hospital's information system to access medical and patient records. Examples of these software programs, sometimes called ‘rounding tools’, have been developed by companies such as MercuryMD (www.mercurymd.com/), Patient Keeper (www.patientkeeper.com/), VISICU (www.visicu.com/index_flash.asp), and Global Care Quest (www.gcq.ucla.edu/index_pc.html).

### BRIEF SUMMARY OF THE INVENTION

In one aspect, the invention provides system for measuring vital signs from multiple patients, typically in an in-hospital setting. The system features a small-scale, body-worn vital sign monitor that includes: i) a sensor configured as a patch that measures electrical and optical signals from a patient; ii) a controller featuring a microprocessor that receives and processes the electrical and optical signals to determine the patient's vital sign information, including blood pressure; and iii) a first short-range wireless component that wirelessly transmits a packet containing the vital sign information to an external receiver. A portable, wireless computer (e.g., a PDA, cellular telephone, or a laptop computer) communicates with the body-worn module. This component includes: i) a second short-range wireless component that receives the vital sign information and displays it; and ii) a long-range wireless transmitter that transmits the vital sign information over a wireless network. The system also includes an Internet-based system that receives the vital sign information from the wireless network, and avails this to medical professionals through an in-hospital information system.

In embodiments, the portable, wireless computer features a software program that processes the packet to determine the body-worn vital sign monitor from which it originated, and a patient associated with the monitor. Typically the packet includes an identifying code, such as a serial number, and the software program includes a database that associates a patient's name with an identifying code. In this case, the Internet-based system can periodically wirelessly transmit contents of the database to the portable, wireless computer.

In a preferred embodiment the patch includes: i) a first adhesive component featuring a first electrode that measures a first electrical signal from the patient; ii) a second adhesive component featuring a second electrode that measures a second electrical signal from the patient; and iii) a third adhesive component, in electrical communication with the first and second adhesive components, featuring an optical system that measures the optical signal from the patient.

In embodiments, the optical system features a light-emitting diode and an optical detector disposed on a same side of a substrate (e.g., a circuit board) to operate in a ‘reflection mode’ geometry. Alternatively, these components can be disposed opposite each other to operate in a ‘transmission mode’ geometry.

The controller typically operates an algorithm (e.g., compiled computer code) configured to process the first and second electrical signals to generate an electrical waveform, and the optical signals to generate an optical waveform. The algorithm then processes the electrical and optical waveforms to calculate a blood pressure value. For example, the controller can determine blood pressure by processing: 1) a first time-dependent feature of the optical waveform; 2) a second time-dependent feature of the electrical waveform; and 3) a calibration parameter determined by another means (e.g., a conventional blood pressure cuff or tonometer).

In embodiments, the third adhesive component further includes a connector configured to connect to a detachable cable that connects to the first and second electrodes. An additional cable can connect the adhesive components to the controller. Alternatively, the third adhesive component can include a first wireless component, and the controller further includes a second wireless component configured to communicate with first wireless component. In yet another embodiment the controller is attached directly to the third adhesive component.

The optical system typically includes a first light-emitting diode that emits radiation (e.g. red radiation) that generates a first optical signal, and a second light-emitting diode that emits radiation (e.g., infrared radiation) that generates a second optical signal. In this case the controller additionally includes an algorithm that processes the first and second optical signals to generate pulse oximetry and heart rate values. In other embodiments the controller features an algorithm that processes the first and second electrical signals to generate an ECG waveform.

In other embodiments the third adhesive component includes a third electrode that measures a third electrical signal from the patient. In this case, the controller includes an algorithm that processes the first, second, and third electrical signals to generate an ECG waveform along with the other vital signs described above.

The invention has many advantages. In particular, it provides a single, low-profile, disposable system that measures a variety of vital signs, including blood pressure, without using a cuff. This and other information can be easily transferred from a patient to a central monitor through a wired or wireless connection. For example, with the system a medical professional can continuously monitor a patient's blood pressure and other vital signs during their day-to-day activities, or while the patient is admitted to a hospital. Monitoring patients in this manner minimizes erroneous measurements due to ‘white coat syndrome’ and increases the accuracy of a blood-pressure measurement. In particular, as described below, one aspect of the invention provides a system that continuously monitors a patient's blood pressure using a cuffless blood pressure monitor and an off-the-shelf mobile communication device. Information describing the blood pressure can be viewed using an Internet-based website, using a personal computer, or simply by viewing a display on the mobile device. Blood-pressure information measured continuously throughout the day provides a relatively comprehensive data set compared to that measured during isolated medical appointments. This approach identifies trends in a patient's blood pressure, such as a gradual increase or decrease, which may indicate a medical condition that requires treatment. The system also minimizes effects of ‘white coat syndrome’ since the monitor automatically and continuously makes measurements away from a medical office with basically no discomfort to the patient. Real-time, automatic blood pressure measurements, followed by wireless transmission of the data, are only practical with a non-invasive, cuffless system like that of the present invention. Measurements can be made completely unobtrusive to the patient.

The system can also characterize the patient's heart rate and blood oxygen saturation using the same optical system for the blood-pressure measurement. This information can be wirelessly transmitted along with blood-pressure information and used to further diagnose the patient's cardiac condition.

The monitor is easily worn by the patient during periods of exercise or day-to-day activities, and makes a non-invasive blood-pressure measurement in a matter of seconds. The resulting information has many uses for patients, medical professional, insurance companies, pharmaceutical agencies conducting clinical trials, and organizations for home-health monitoring.

Having briefly described the present invention, the above and further objects, features and advantages thereof will be recognized by those skilled in the pertinent art from the following detailed description of the invention when taken in conjunction with the accompanying drawings.

### BRIEF DESCRIPTION OF SEVERAL VIEWS OF THE DRAWINGS

FIG. 1A shows a semi-schematic view of a vital sign-monitoring system according to the invention featuring a disposable patch sensor connected to a body-worn monitor that, in turn, communicates with an external wireless PDA;

FIG. 1B shows a top view of the disposable patch sensor of FIG. 1A;

FIG. 2 shows a semi-schematic view of the wireless PDA of FIG. 1A connected to multiple body-worn monitors in, e.g., a hospital setting;

FIG. 3 shows a graph of time-dependent optical and electrical waveforms collected by the body-worn module of FIG. 1A;

FIG. 4 shows a screen shot of a user interface deployed on the wireless PDA of FIG. 1A; and

FIG. 5 shows an Internet-based system used to route information from the PDA to an in-hospital information system.

### DETAILED DESCRIPTION OF THE INVENTION

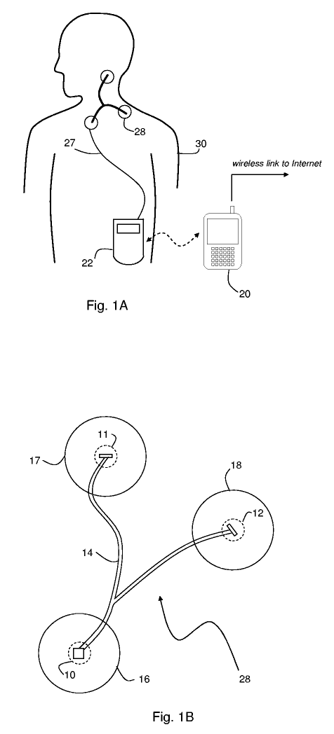

FIGS. 1A and 1B show, respectively, a body-worn vital sign monitor 22 that connects through a cable 27 to a disposable patch sensor 28 attached to a patient 30. The patch sensor 28 measures optical and electrical waveforms, described in detail below with reference to FIG. 3, that the body-worn monitor 22 receives and processes to calculate the patient's blood pressure and other vital signs. Once this information is calculated, the body-worn monitor 22 sends it to an external, wireless PDA 20 through a wireless link (e.g., a Bluetooth connection). The PDA 20 can process and display the information and then transmit it wirelessly over a nation-wide network (e.g. a CDMA network) to an Internet-accessible website or hospital information system, as described in more detail below with reference to FIG. 5.

Preferably the patch sensor 28 attaches to a region near the patient's neck, chest, ear, or to any other location that is near the patient's head and proximal to an underling artery. Typically the patient's head undergoes relatively little motion compared to other parts of the body (e.g., the hands), and thus attaching the patch sensor 28 to these regions reduces the negative affects of motion-related artifacts.

FIG. 1B shows the disposable patch sensor 28 that features primary 11 and reference 12 electrodes and an optical system 10 operating in concert as described below to measure vital signs from a patient 30. The electrodes 11, 12 and optical system 10 each attach to the patient's skin using a separate adhesive pad 16, 17, 18, and connect to each other using a Y-shaped cable 14. During operation, the primary 11 and reference 12 electrodes detect electrical impulses, similar to those used to generate a conventional ECG, from the patient's skin. Each heartbeat generates a unique set of electrical impulses. Concurrently, the optical system 10 measures an optical waveform by detecting a time-dependent volumetric change in an underlying artery caused by blood flow following each heartbeat. The optical waveform is similar to an optical plethysmograph measured by a pulse oximeter. During operation, the body-worn monitor 22 receives the electrical impulses and converts these to an electrical waveform (e.g., an ECG), and is described in more detail in U.S. patent application Ser. No. 10/906,314, filed Feb. 14, 2005 and entitled PATCH SENSOR FOR MEASURING BLOOD PRESSURE WITHOUT A CUFF, the contents of which are incorporated herein by reference. The body-worn monitor includes a microprocessor that runs an algorithm to process the electrical and optical waveforms to measure vital signs, such as pulse oximetry, heart rate, ECG, and blood pressure.

For the purposes of measuring blood pressure as described herein, the primary 11 and reference 12 electrodes only need to collect electrical signals required to generate an electrical waveform found in a 2-lead ECG. These electrodes can therefore be placed on the patient at positions that differ from those used during a standard multi-lead ECG (e.g., positions used in ‘Einthoven's Triangle’).

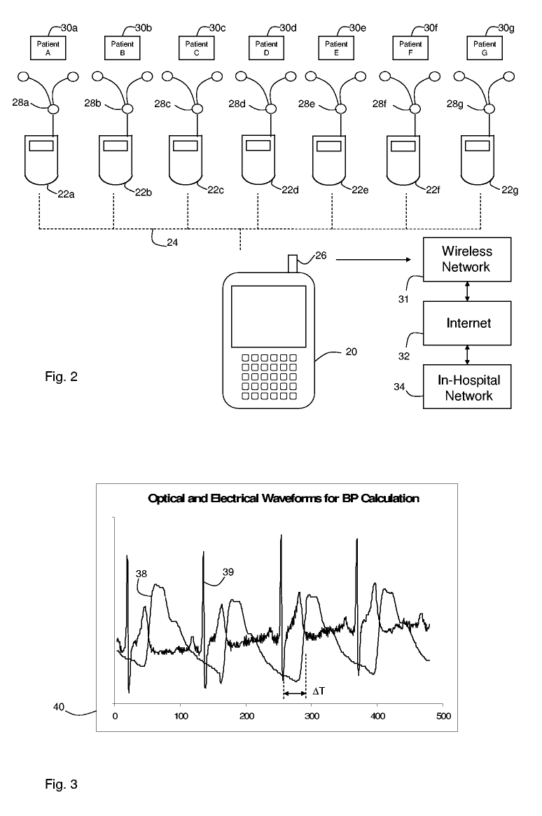

FIG. 2 shows how a single wireless PDA 20 operates in a hospital environment to collect vital sign information from a set of body-worn monitors 22a-g, each associated with a separate patch sensor 28a-g attached to a unique patient 30a-g. For example, each patient 30a-g wearing a body-worn monitor 22a-g and patch sensor 28a-g can be located within a unique hospital room. A medical professional making ‘rounds’ sequentially enters each room and downloads the patient's most recent vital sign information from each body-worn monitor 22a-g through a short-range wireless connection (using, e.g., a pair of matched Bluetooth™ transceivers). In this case, each body-worn monitor 22a-g sends information in a packet that includes a header describing a serial number of the monitor, and a payload describing the vital sign information. The PDA 20, in turn, includes a database that is typically downloaded wirelessly from a central server. The database associates the serial number and the vital sign information with the patient's name. Once the vital sign information is collected from each patient 22a-g, the PDA 20 formats it accordingly and sends it using an antenna 26 through a nation-wide wireless network 31 to a computer system on the Internet 32. The computer system then sends the information through the Internet 32 to an in-hospital network 34 (using, e.g., a frame-relay circuit or VPN). From there, the information is associated with a patient's medical records, and can be accessed at a later time by a medical professional.

FIG. 3 shows a graph 40 that plots both the optical 38 and electrical 39 waveforms generated by, respectively, the electrodes and optical system in the disposable patch sensor. Both waveforms include multiple ‘pulses’ each corresponding to an individual heartbeat. Following the heartbeat, electrical impulses travel essentially instantaneously from the patient's heart to the electrodes, which detect it to generate a pulse in the electrical waveform 39. At a later time, a pressure wave induced by the same heartbeat propagates through the patient's arteries, which are elastic and increase in volume due to the pressure wave. Ultimately the pressure wave arrives at a portion of the artery underneath the optical system, where light-emitting diodes and a photodetector detect it by measuring a time-dependent change in optical absorption to generate the optical waveform 38. The propagation time of the electrical impulse is independent of blood pressure, whereas the propagation time of the pressure wave depends strongly on pressure, as well as mechanical properties of the patient's arteries (e.g., arterial size, stiffness). The microprocessor runs an algorithm that analyzes the time difference ΔT between the arrivals of these signals, i.e. the relative occurrence of pulses in the optical 38 and electrical 39 waveforms as measured by the patch sensor. Calibrating the measurement (e.g., with a conventional blood pressure cuff or tonometer) accounts for patient-to-patient variations in arterial properties, and correlates ΔT and other properties of the waveforms to both systolic and diastolic blood pressure. This results in a calibration table. During an actual measurement, the calibration source is removed, and the microprocessor analyzes ΔT along with other properties of the optical and electrical waveforms and the calibration table to calculate the patient's real-time blood pressure.

In one embodiment, for example, the microprocessor ‘fits’ the optical waveform using a mathematical function that accurately describes the waveform's features, and an algorithm (e.g., the Marquardt-Levenberg algorithm) that iteratively varies the parameters of the fitting function until it best matches the time-dependent features of the waveform. In this way, blood pressure-dependent properties of the waveform, such as its width, rise time, fall time, and area, can be calibrated as described above. After the calibration source is removed, the patch sensor measures these properties along with ΔT to determine the patient's blood pressure. Alternatively, the waveforms can be filtered using mathematical techniques, e.g. to remove high or low frequency components that do not correlate to blood pressure. In this case the waveforms can be filtered using well-known Fourier Transform techniques or simple smoothing algorithms to remove unwanted frequency components, and then processed as described above.

Methods for processing the optical and electrical waveform to determine blood pressure are described in the following co-pending patent applications, the entire contents of which are incorporated by reference: 1) CUFFLESS BLOOD-PRESSURE MONITOR AND ACCOMPANYING WIRELESS, INTERNET-BASED SYSTEM (U.S. Ser. No. 10/709,015; filed Apr. 7, 2004); 2) CUFFLESS SYSTEM FOR MEASURING BLOOD PRESSURE (U.S. Ser. No. 10/709,014; filed Apr. 7, 2004); 3) CUFFLESS BLOOD PRESSURE MONITOR AND ACCOMPANYING WEB SERVICES INTERFACE (U.S. Ser. No. 10/810,237; filed Mar. 26, 2004); 4) VITAL-SIGN MONITOR FOR ATHLETIC APPLICATIONS (U.S. Ser. No. ______; filed Sep. 13, 2004); 5) CUFFLESS BLOOD PRESSURE MONITOR AND ACCOMPANYING WIRELESS MOBILE DEVICE (U.S. Ser. No. 10/967,511; filed Oct. 18, 2004); and 6) BLOOD PRESSURE MONITORING DEVICE FEATURING A CALIBRATION-BASED ANALYSIS (U.S. Ser. No. 10/967,610; filed Oct. 18, 2004); 7) PERSONAL COMPUTER-BASED VITAL SIGN MONITOR (U.S. Ser. No. 10/906,342; filed Feb. 15, 2005); and 8) PATCH SENSOR FOR MEASURING BLOOD PRESSURE WITHOUT A CUFF (U.S. Ser. No. 10/906,315; filed Feb. 14, 2005).

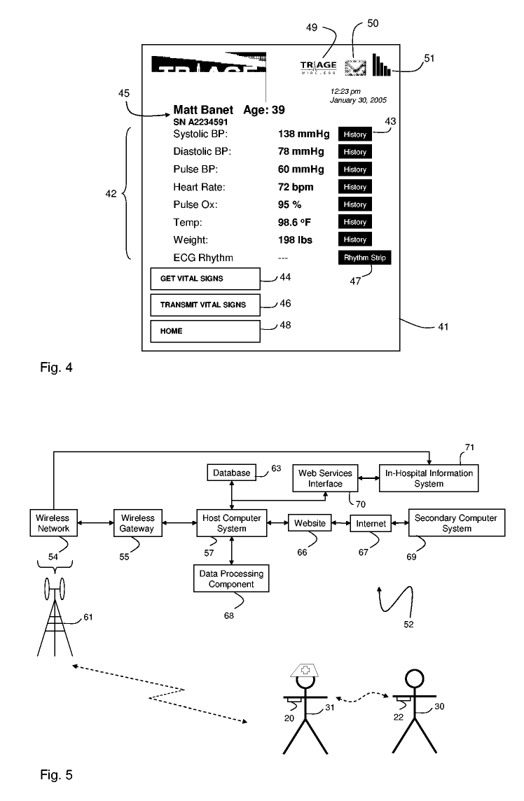

FIG. 4 shows a screen shot of a graphical user interface (GUI) 41, rendered on the wireless PDA, which displays patient information 45 and vital sign information 42. For example, a medical professional (e.g. a nurse) can turn on the PDA before making rounds at a hospital; this process loads the GUI 41. When the nurse enters a hospital room, the PDA detects a short-range wireless signal indicating the presence of a patient wearing a body-worn vital sign monitor, described above. The PDA displays a serial number associated with the monitor, along with the patient's name, in the patient information 45. The nurse then depresses a ‘Get Vital Signs’ button 44 on the GUI 41. This initiates a wireless serial link with the body-worn monitor, and then downloads a set of vital signs collected recently by the patch sensor. As shown in the figure, this information includes:
1) Systolic blood pressure
2) Diastolic blood pressure
3) Pulse blood pressure
4) Heart rate
5) Pulse oximetry
6) Temperature
7) Weight
8) ECG ‘rhythm strip’ (e.g., the electrical waveform shown in FIG. 3)

Note that for the above-mentioned information, temperature is measured with a conventional temperature sensor embedded in the patch sensor. Weight is measured at an earlier time when the patient steps on a scale that includes a short-range wireless transceiver that connects to a matched transceiver within the body-worn unit. Such a system, for example, is described in the pending patent application entitled ‘SMALL-SCALE, VITAL-SIGNS MONITORING DEVICE, SYSTEM AND METHOD’, U.S. Ser. No. 10/907,440, filed Mar. 31, 2005, the contents of which are incorporated herein by reference.

In addition to collecting the patient's most recent vital sign information 42, the nurse can depress a ‘History’ button 43 to collect historical values of a particular vital sign. Once collected, these values can be plotted in a variety of graphical formats, such as a time-dependent or histogram format. Similarly, the GUI 41 includes a ‘Rhythm Strip’ button 47 that, once depressed, renders and analyzes a graphical ECG rhythm strip, similar to the electrical waveform shown in FIG. 3.

Once the nurse collects the patient's most recent or historical vital sign information, a ‘Transmit Vital Signs’ button 46 is depressed to transmit this information over a wireless network, such as a nation-wide (e.g., a CDMA network) or in-hospital wireless network (e.g. an 802.11-based network), to the hospital's information system. This information can then be accessed at a later time by any relevant medical personnel associated with the patient or hospital.

The GUI 41 also includes other tools for managing information, such as a link 49 to a web page on the Internet, a link 50 to a email program, a button 48 that connects the nurse to a home page of the GUI that includes links to other data-processing functions, and an icon 51 that describes the strength of the wireless signal.

FIG. 5 shows a preferred embodiment of an Internet-based system 52 that operates in concert with the body-worn unit 22 to send information from a patient 30 to an in-hospital information system 71. Using a wireless PDA 20 operating a GUI such as that shown in FIG. 4, a medical professional 31 collects vital sign information from the patient's body-worn unit 22 through a short-range wireless connection. The wireless PDA 20 then sends the information through a wireless network 54 to a web site 66 hosted on an Internet-based host computer system 57. The wireless network can be a nation-wide wireless network or a local wireless network. A secondary computer system 69 accesses the website 66 through the Internet 67. A wireless gateway 55 connects to the wireless network 54 and receives data from one or more wireless PDAs 20, as discussed below. The host computer system 57 includes a database 63 and a data-processing component 68 for, respectively, storing and analyzing the data. The host computer system 57, for example, may include multiple computers, software pieces, and other signal-processing and switching equipment, such as routers and digital signal processors. The wireless gateway 55 preferably connects to the wireless network 54 using a TCP/IP-based connection, or with a dedicated, digital leased line (e.g., a frame-relay circuit or a digital line running an X.25 or other protocols). The host computer system 57 also hosts the web site 66 using conventional computer hardware (e.g. computer servers for both a database and the web site) and software (e.g., web server and database software). To connect to the in-hospital information system 71, the host computer system 57 typically includes a web services interface 70 that sends information using an XML-based web services link to a computer associated with the in-hospital information system 71. Alternatively, the wireless network 54 may be an in-hospital wireless network (e.g., a network operating Bluetooth™, 802.11a, 802.11b, 802.1g, 802.15.4, or ‘mesh network’ wireless protocols) that connects directly to the in-hospital information system 71. In this embodiment, a nurse working at a central nursing station can quickly view the vital signs of the patient using a simple computer interface.

To view information remotely, the patient or medical professional can access a user interface hosted on the web site 66 through the Internet 67 from a secondary computer system 69, such as a Internet-accessible home computer. The system 53 may also include a call center, typically staffed with medical professionals such as doctors, nurses, or nurse practioners, whom access a care-provider interface hosted on the same website 66.

During typical operation, the patient continuously wears the body-worn monitor 22 and its associated patch sensor system during their hospital stay, which is typically a period of time ranging from a few hours to weeks.

The body-worn can optionally be used to determine the patient's location using embedded position-location technology (e.g., GPS, network-assisted GPS, or Bluetooth™, 802.11-based location system). In situations requiring immediate medical assistance, the patient's location, along with relevant vital sign information, can be relayed to emergency response personnel.

In a related embodiment, the wireless PDA may use a ‘store and forward’ protocol wherein one of these devices stores information when the wireless device is out of wireless coverage, and then sends this information to the wireless device when it roams back into wireless coverage.

In still other embodiments, electronics associated with the body-worn monitor (e.g., the microprocessor) are disposed directly on the patch sensor, e.g. on a circuit board that supports the optical system. In this configuration, the circuit board may also include a display to render the patient's vital signs. In another embodiment, a short-range radio (e.g., a Bluetooth™, 802.15.4, or part-15 radio) is mounted on the circuit board and wirelessly sends information (e.g., optical and electrical waveforms; calculated vital signs such as blood pressure, heart rate, pulse oximetry, ECG, and associated waveforms) to an external controller with a matched radio, or to a conventional cellular telephone or wireless personal digital assistant. Or the short-range radio may send information to a central computer system (e.g., a computer at a nursing station), or though an internal wireless network (e.g. an 802.11-based in-hospital network). In yet another embodiment, the circuit board can support a computer memory that stores multiple readings, each corresponding to a unique time/date stamp. In this case, the readings can be accessed using a wireless or wired system described above.

In still other embodiments, the patch sensor can include sensors in addition to those described above, e.g. sensors that measure motion (e.g. an accelerometer) or other properties.

Still other embodiments are within the scope of the following claims.

What is claimed is:

1. A system for measuring vital signs from a plurality of patients, comprising:
a body-worn vital sign monitor comprising: i) a sensor configured as a patch that measures electrical and optical signals from a patient; ii) a controller comprising a microprocessor that receives and processes the electrical and optical signals to determine the patient's vital sign information, including blood pressure; and iii) a first short-range wireless component that wirelessly transmits a packet comprising the vital sign information to an external receiver;
a portable, wireless computer comprising: i) a second short-range wireless component that receives the vital sign information and displays it; and ii) a long-range wireless transmitter that transmits the vital sign information over a wireless network; and
an Internet-based system that receives the vital sign information from the wireless network.

2. The system of claim 1, wherein the portable, wireless computer comprises a software program that processes the packet to identify the body-worn vital sign monitor from which it originated.

3. The system of claim 2, wherein the software program comprises a database that associates a patient's name with an identifying code comprised by the packet.

4. The system of claim 3, wherein the Internet-based system further comprises a software component that sends contents of the database to the portable, wireless computer.

5. The system of claim 1, wherein the portable, wireless computer further comprises a software program that detects a body-worn vital sign monitor, and a user interface that displays a patient associated with the body-worn monitor.

6. The system of claim 1, wherein the Internet-based system further comprises an interface to a hospital information system.

7. The system of claim 6, wherein the interface is a web services interface.

8. The system of claim 1, wherein the portable, wireless computer is a personal digital assistant, cellular telephone, or a laptop computer.

9. The system of claim 1, wherein the patch comprises:
a first adhesive component comprising a first electrode that measures a first electrical signal from the patient;
a second adhesive component comprising a second electrode that measures a second electrical signal from the patient; and
a third adhesive component, in electrical communication with the first and second adhesive components, comprising an optical system that measures the optical signal from the patient.

10. The system of claim 9, wherein the optical system comprises a light-emitting diode and an optical detector.

11. The system of claim 10, wherein the optical system further comprises a substrate, and the light-emitting diode and optical detector are disposed on a same side of the substrate.

12. The system of claim 11, further comprising an optical detector aligned to detect radiation first emitted from the light-emitting diode and then reflected from the patient's skin to generate the optical waveform.

13. The system of claim 1, wherein the controller further comprises an algorithm configured to process the first and second electrical signals to generate an electrical waveform.

14. The system of claim 13, wherein the controller further comprises an algorithm configured to process the optical signal to generate an optical waveform.

15. The system of claim 14, wherein the controller further comprises an algorithm that processes the electrical and optical waveforms to calculate a blood pressure value.

16. The system of claim 15, wherein the controller further comprises an algorithm that determines blood pressure by processing: 1) a first time-dependent feature of the optical waveform; 2) a second time-dependent feature of the electrical waveform; and 3) a calibration parameter.

17. A system for measuring vital signs from a plurality of patients, comprising:
a body-worn vital sign monitor, comprising:
a sensor configured as a patch comprising: i) a first adhesive component comprising a first electrode that measures a first electrical signal from the patient; ii) a second adhesive component comprising a second electrode that measures a second electrical signal from the patient; and iii) a third adhesive component, in electrical communication with the first and second adhesive components, comprising an optical system that measures the optical signal from the patient;
a controller comprising: i) a microprocessor that receives and processes the electrical and optical signals to determine the patient's vital sign information, including blood pressure;
and ii) a first short-range wireless component that wirelessly transmits a packet comprising the vital sign information to an external receiver;
a portable, wireless computer comprising: i) a second short-range wireless component that receives the vital sign information and displays it; and ii) a long-range wireless transmitter that transmits the vital sign information over a wireless network; and
an Internet-based system that receives the vital sign information from the wireless network.

18. A system for measuring vital signs from a plurality of patients, comprising:
a body-worn vital sign monitor, comprising: i) a sensor configured as a patch that measures electrical and optical signals from a patient; ii) a controller comprising a microprocessor that receives and processes the electrical and optical signals to determine the patient's vital sign information, including blood pressure; and iii) a first short-range wireless component that wirelessly transmits a packet comprising the vital sign information and an identifying code to an external receiver;
a portable, wireless computer comprising: i) a second short-range wireless component that receives the vital sign information and the identifying code, and in response displays the vital sign information and a patient associated with the identifying code; and ii) a long-range wireless transmitter that transmits the vital sign information over a wireless network; and an Internet-based system that receives the vital sign information from the wireless network.
